(19) United States
(12) Patent Application Publication
(10) Pub. No.: US 2006/0177891 A1
(43) Pub. Date: Aug. 10, 2006

(54) Fiber optic bio-sensor

(76) Inventors: Anil Kishen, Singapore (SG); Shelly John Mechery, Singapore (SG); Chu-Sing Lim, Singapore (SG); Anand Krishna Asundi, Singapore (SG)

Correspondence Address: LAWRENCE Y.D. HO & ASSOCIATES PTE LTD, 30 BIDEFORD ROAD, #07-01, THONGSIA BUILDING, SINGAPORE, 229922, SG

(21) Appl. No.: 10/510,043

(22) PCT Filed: Mar. 28, 2003
(86) PCT No.: PCT/SG03/00063

§ 371 Date: Jun. 15, 2005

(30) Foreign Application Priority Data
Apr. 3, 2002 (SG) 200201691-3

(51) Int. Cl. C12Q 1/04; C12M 1/34; G01J 3/42

(52) U.S. Cl. 435/34; 435/287.1; 356/319

(57) ABSTRACT

A sensor for sensing and monitoring a property associated with transformation of a biochemical analyte by a micro-organism, the sensor comprising a glass permeable coating applied to an unclad portion (14) of a fibre optic member (13). The coating has a transformable precursor impregnated into it, the precursor being specifically metabolisable by one or more targeted organisms. When the sensor is placed in contact with a sample in a container (15) contacting an active targeted micro-organisms, the precursor is transformed by the micro-organisms to produce a spectroscopically detectable indicator of the property of the analyte. Spectroscopic information may be analysed by a computer program to provide an overall index of microbiological activity for the targeted micro-organism. The invention extends to a method of producing a sensor and a method of identifying the presence of a targeted micro-organism.

### FIELD OF THE INVENTION

This invention relates generally to devices and methods for identifying the presence, and monitoring the activity, of a particular microorganism, including bacteria, in a sample. More specifically, this invention is associated with a device and methods based on the use of optical fibers applied to the detection, identification, and monitoring of a particular microorganism or species or group of microorganisms in a sample including an environmental-, industrial- or human-derived sample.

### BACKGROUND OF THE INVENTION

The isolation and identification of one or more microorganisms present in a sample has long been of great importance in a wide range of situations. The fields in which the need for such procedures may arise include medical diagnosis and treatment, military applications (e.g. biological warfare), security agencies, agriculture, food processing and water quality assessment and control. In the field of medical diagnosis and treatment of both people and animals, time may be of the essence in detecting and characterising a microorganism in order to facilitate appropriate chemotherapeutic intervention. There are many other instances where the application of a rapid and relatively cheap test for determining the presence of microorganisms may be of great assistance. One example of such a situation arises in the case of Streptococcus mutans in saliva which is a predisposing factor to the development of dental caries. Early identification and treatment may lead to a significant decrease in tooth and gum disease.

Traditionally microorganisms such as bacteria have been identified by methods which include cell culture, microscopy and more recently immunoassay and nucleic acid probes. Culturing bacteria requires the deposition of a sample onto a suitable culture medium such as an agar plate. The combination of sample and medium is incubated in a suitable environment and after a period of time such as 24-48 hours, colonies may be harvested and subjected to identifying tests. If mixed colonies of bacteria are grown it may be necessary to resample the growth and repeat the process to obtain separate colonies of individual bacteria.

Identification has typically required subjecting the cells from the cultured colony to one or more characterising tests. These microbiological techniques often require optimum specimen quality to ensure an accurate analytical result. These current techniques are mostly only qualitative, and are therefore difficult for monitoring bacterial activity quantitatively.

Bacterial structure may be a significant consideration in choosing appropriate tests. Cell walls of bacteria may comprise three morphologically defined layers. The outermost layer may consist of lipids, polysaccharides and proteins. This layer distinguishes gram negative bacteria from gram positive bacteria. The gram positive bacteria lack this outermost layer thereby providing the basis for one of the most fundamental tests in microboiology.

Development of identification methods has been established from past microbiological studies identifying specific biochemical reactions that some species or groups of microorganisms display. These specific microorganism mediated biochemical reactions form the basis of traditionally practised laboratory methods for identifying and characterising species and strains.

With evolving technology, more sophisticated procedures such as fluorescent techniques and nucleic acid identification have also been established. There has always been a great interest in rapid identification of disease related microorganisms. Towards this end, researchers have applied optical techniques such as Fourier Transfer Infrared (FTIR) spectroscopy and fluorescence-based techniques. The former technique works on the principle of obtaining complex finger prints from bacterial strains based on constituents of the cell wall. The latter technique typically employs fluorescing protein markers to monitor bacterial activity. The FTIR techniques rely on algorithms and spectral analysis that compare corrugated spectroscopic patterns to identify bacterial strains. This method requires the use of dry specimens (after controlled heating in an oven) for analysis. Consequently, this technique is complex, time consuming and cannot be used directly on a patient. A fluorescence based technique is very specific for a bacterial reaction but requires an extended preparatory and characterisation phase before the application of the sensor to the identification of a bacterial strain. Application of these methods may be difficult, expensive, time consuming and therefore not well adapted for routine use.

U.S. Pat. No. 5,496,700 to Ligler et al describes an optical immunoassay for microbial analytes using non-specific dyes. Microorganisms in a sample are all stained using a non-specific dye. The stained sample is placed in contact with an optical wave guide which is coated with a capture molecule. A sample suspected of containing a microbial analyte is mixed with a dye and then exposed to the solid support material which has an attached capture molecule specific for the suspected microbial analyte. In the preferred embodiment of the invention, the dye is fluorescent. Fluorescent emission technology utilises the ability of some compounds to absorb light of a particular wave length and emit light of a different specific wave length. The use of such methods usually requires considerable preparation time and cost in production.

Ligler's method relies on the fixation of a capture molecule to the wave guide device. In use a positive test will result from formation of a complex including a dye, the microbial analyte and the capture molecule. This test therefore necessitates an initial dying step and the production of an analyte specific capture molecule which must be placed on the wave guide. The steps are relatively complex and sophisticated and require the foundation step of producing an analyte specific capture molecule and fixing it to the wave guide. The method and device is unlikely to be of particular use in vivo.

U.S. Pat. No. 6,256,522 to Schultz discloses a sensor for continuous monitoring of biochemicals and a related method. The disclosure relates to a sensor capsule having a processing chamber defined by a wall which allows the passage of an analyte. Material capable of interacting with the analyte is contained in the chamber. A light source, which may be an optical fibre, causes light to impinge on a translucent portion of the chamber. Responsive fluorescent light is generated and emitted and may be processed to determine concentration of an analyte. The disclosure does not describe an easy and effective method for determining the presence of a microorganism such as a type of bacteria, in a sample and relates to a relatively complex, sophisticated and costly piece of equipment.

It would be advantageous to provide a quick and effective method of determining the presence of, and monitor, microorganisms such as bacteria and preferably identifying the group, species or type of microorganism.

The reference to any prior art in this specification is not, and should not be taken as, an acknowledgment or any form of suggestion that that prior art forms part of the common general knowledge in any country.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In one form, although it need not be the only or indeed the broadest form, the invention resides in a sensor for sensing at least one property associated with transformation of a biochemical analyte by a microorganism, said sensor comprising:

at least one fibre optic member having at least one unclad portion;

a coating applied to the at least one unclad portion;

a precursor associated with the coating, said precursor transformable by at least one microorganism;

wherein

transformation of the precursor produces a spectroscopically detectable indicator of the at least one property.

The microorganism may be a prokaryote or eukaryote. A eukaryote includes a mammalian cell including a human cell, or other animal cell such as an insect cell, yeast, fungus or amoebic cell. A prokaryote is particularly preferred and includes all genera and/or species of bacteria.

The unclad portion of the fibre optic member is preferably a declad portion. The fibre optic member may have a plurality of unclad portions. The plurality of unclad portions may be contiguous, spaced or a combination of the two. The sensor may comprise two or more separate fibre optic members. The separate optic fibres may be substantially parallel.

Preferably the at least one fibre optic member has a first end adapted to receive light from a light source. The at least one fibre optic member may have a second outlet end adapted to co-operate with analysis means to determine the presence of the spectroscopically detectable indicator. The at least one fibre optic member may be formed in a “Y” shaped configuration including three ends, a first end adapted to receive light from a light source, a second end adapted to co-operate with analysis means to determine the presence of the spectroscopically detectable indicator and a reflective end for reflecting light, the reflective end located on the equivalent and the declad portion of the lower most arm of the “Y” shape.

The coating is preferably a glass film. Suitably the glass film is both porous and thin. The precursor may be immobilised within the coating. Alternatively or additionally the precursor may be immobilised on a surface of the coating. The precursor may comprise one or more of D-mannitol, carbol fuchsine, methylene blue, sucrose or other suitable compound.

The precursor may be selected to identify the presence of a single microorganism species or, perhaps, variety. Alternatively, the precursor may be selected to identify two or more microorganism species or varieties or a group thereof. Most preferably the precursor is selected to identify one or more bacteria.

Transformation of the precursor may produce the spectroscopically detectable indicator directly. Alternatively, transformation of the precursor may result in a product which cooperates with one or more adjunctive compounds to produce the spectroscopically detectable indicator.

“Spectroscopically detectable” may include optically detectable. The spectroscopically detectable indicator is preferably substantially formed in a zone of evanescent light waves adjacent to an outer surface of a fibre optic core of the at least one unclad region. The spectroscopically detectable indicator may, in operation, be illuminated by evanescent light waves.

In a second aspect the invention resides in a sensor system for sensing at least one property associated with transformation of a precursor by one or more microorganisms, said system comprising:

a fibre optic member having at least one unclad portion of optic fibre;

a coating applied to the at least one unclad portion;

a precursor associated with the coating, said precursor transformable by at least one microorganism; and

a light source adapted to co-operate with a first end of the fibre optic member;

monitoring means adapted to co-operate with the unclad portion to detect an indicator signal;

wherein

transformation of the precursor by the one or more microorganisms produces the indicator signal.

In a third aspect the invention resides in a method of producing a sensor, said method comprising the steps of:

decladding one or more sections of a core of a fibre optic member;

applying a coating to the one or more sections, said coating immobilising a precursor to a spectroscopically detectable indicator, the precursor transformable to the detectable indicator by the activity of one or more microorganisms.

In a fourth aspect the invention resides in a method of identifying the presence of at least one type of microorganism, the method comprising the steps of:

activating a light source in co-operative relationship to a first end of a sensor as herein described;

monitoring the electromagnetic out-put from a coated, unclad section;

locating the sensor with its coated, unclad section in contact with a sample; and

analysing the electromagnetic output to determine the presence of the at least one type of microorganism.

Monitoring the electromagnetic output may comprise spectroscopically monitoring the electromagnetic output. The electromagnetic output may be light output.

Preferably the electromagnetic output is monitored through a second end of the sensor.

Locating the sensor may include immersing the sensor in a liquid sample. Locating the sensor may alternatively or further include placing the unclad section in contact with living tissue.

Analysing the electromagnetic output preferably comprises absorption analysis to identify the wave length of peak absorption of electromagnetic output.

Analysing the electromagnetic output may also include operating a programmable device programmed to receive digital information from a spectroscope and provide an analysis of results.

The light source may be any suitable apparatus and may comprise a tungsten-halogen lamp. A xenon-arc lamp may also be used.

Preferably the monitoring means includes spectroscopic analysis means. The spectroscopic analysis means may include a processing system including at least:
a) an input for receiving input data from a spectroscope;
b) a store for storing identification data for one or more organisms; and
c) a processor, the processor being adapted to:
   1) compare the input data to the identification data; and
   2) generate a report indicating presence and type of one or more microorganisms.

The processor may be programmed to store sample identification data which may include information such as sample origin, time and date of collection.

### BRIEF DESCRIPTION OF THE DRAWINGS

FIG. 1 shows a schematic representation of a first embodiment of a sensor of the present invention.

FIG. 2 shows a schematic representation of a second embodiment of a sensor of the present invention.

FIG. 3 shows a side schematic view of a sensor probe.

FIG. 4A shows an example of a spectrum of light production from a light source.

FIG. 4B represents the evanescent wave distribution at the core-cladding interface.

FIG. 4C represents an example of a transmission spectrum from a sensor system.

FIG. 5 shows a graph for light absorption over time for a sensor of the present invention.

FIG. 6 shows a graph for variation in intensity of the absorbance valley when using a sensor system of the present invention.

### DETAILED DESCRIPTION OF THE INVENTION

The present invention is directed to a fibre optic microorganism sensor which may be used to detect and monitor one or more specific activities of microorganisms.

The term “sample” includes inter alia a biological, industrial and/or environmental sample. The term “biological sample” is used in its broadest sense and includes a sample of tissue or cells from a tissue or organ isolated by, for example, surgical intervention, biopsy, lymph fluid, exudate (eg. pus, discharge), waste products (eg. urine, faeces), blood collection procedures or invasive or passive collection procedures. The expression “blood collection procedures” encompass serum, plasma and blood fractions. Furthermore, a biological sample may comprise cells maintained in vitro culture or suspension. A biological sample is, therefore, a collection or population of cells which may comprise a single cell type or comprise a mixed population of two or more cell types. An environmental sample includes an industrial sample and encompasses any location such as a water supply, food-handling areas, terrestrial locations, waste-dumps, commercial areas etc.

As contemplated herein microorganisms include prokaryotic and eukaryotic cells. Prokaryotic cells include any bacterial or microbial cell such as present in an environmental or biological sample. Such prokaryotic organisms include Pseudomonas sp., E. coli, Enterobacter sp., Salmonella sp., Klebsiella sp., Acetobacter sp., Porphroymonas sp., Staphylococcous sp., Streptococcus sp., Bacillus sp., Proteus sp., Helicobacter sp., Campylobacter sp. or Legionella sp. amongst many others. Viruses include hepatitis virus, a retrovirus, an AIDS virus (e.g. HIV), foot and mouth disease virus or polio virus amongst many others. Eukaroytic cells include eukaryotic organisms such as yeast, fungi, amoeba, and other single cell organisms as well as cells from higher plants or animals.

Bacteria may include E. coli stains such as but not limited to, WA803, WA802, RR1, Q359, Q538, P2392, NM621, NM554, NM477, MC4100, MC1061, DL538, DB1316, CSH18, CES200, C600hfi, C600, BNN102, BNN93, BL21(DE3), and BHB2690. Other suitable bacteria may include but are not limited to the following bacteria, Aminobacterium mobile DSM 12262, Aminomonas paucivorans DSM 12260, Asaia bogorensis JCM 10569, Bacteroides thetaiotaomicron BTX, Burkholderia kururiensis JCM 10599, Desulfovibrio dechloracetivorans SF3, Escherichia coli HS(pFamp)R, Kocuria rhizophila DSM 11926, Methylobacterium mesophilicum AM24, Mycobacterium avium MAC 511, Mycobacterium avium MAC 101, Phormidium corium, Pseudomonas aeruginosa ERC1, Pseudomonas aeruginosa HER-1001, Pseudomonas aeruginosa HER-1002, Pseudomonas aeruginosa HER-1010, Pseudomonas aeruginosa HER-1009, Pseudomonas aeruginosa HER-1016, Pseudomonas aeruginosa HER-1017, Pseudoxanthomonas broegbemensis DSM 12573, Ralstonia gilardii LMG 5886, Shewanella frigidimarina ACAM 591, Shewanella gelidimarina ACAM 456, Streptococcus pneumoniae MS22, Streptococcus pneumoniae Fi10, Streptococcus pneumoniae 51702, Streptococcus pneumoniae TW31, Streptococcus pneumoniae TW17, Thiomicrospira frisia JB-A2, Thiomicrospira kuenenii JB-A1, Treponema lecithinolyticum OMZ 685, Treponema maltophilum BR, Treponema maltophilum PNA1, Treponema maltophilum H02A, Ureaplasma urealyticum. Still other microorganisms may include but are not limited to the following fungal cells Hyphodontia australis 231Kluyveromyces lactis CK56-7A, Kluyveromyces lactis CW64-1C, Prosthemium asterosporum A1, Prosthemium betulinum B1 Saccharomyces cerevisiae 1A-H19 [psi-], Saccharomyces cerevisiae 5V-H19 [psi-], Saccharomyces cerevisiae 1-5V-H19, Saccharomyces cerevisiae PS-5V-H19, Saccharomyces cerevisiae C10B-H49, Saccharomyces cerevisiae 9V-H70 [PIN+], Saccharomyces cerevisiae 4V-H73, Saccharomyces cerevisiae 17G-H73, Saccharomyces cerevisiae 3B-H72, Saccharomyces cerevisiae DL1, Saccharomyces cerevisiae GW226, Saccharomyces cerevisiae JM43-GD7, Saccharomyces cerevisiae MCC318, Saccharomyces cerevisiae NB39-5D, Saccharomyces cerevisiae NGB108, Saccharomyces cerevisiae PTH43, Saccharomyces cerevisiae PTH352, Saccharomyces cerevisiae PTY11, Saccharomyces cerevisiae TF112, Saccharomyces cerevisiae TWM1041, Saccharomyces kluyveri GRY1175, Saccharomyces kluyveri MCC328 and Saccharomyces kluyveri NB180.

A eukaryotic organism includes a yeast, fungus, amoeba, parasite, insect and the like.

Preferably the utility of the present system arises from the capacity for tailoring the system to one or more wide range of types of microorganisms including bacteria and specific strains of bacteria, by the selection of a relevant biochemical reagent or precursor for incorporation in the sensor region of the fibre optic system. This provides a device which may perform measurements rapidly and qualitatively and even quantitatively. The system provides a safe means to analyse biological materials and biochemical reactions.

The method of the present invention may be conveniently classed into three phases being:
1) a fibre optic transduction phase;
2) a biochemical recognition phase; and
3) a spectroscopic analytical phase.

The fibre optic transduction phase arises from transmission of light through an optical fibre during which total internal reflection takes place at an interface between the core of the fibre optic member and an external cladding. During each total internal reflection, a certain portion of electromagnetic radiation or wave penetrates the cladding. This wave is called the evanescent wave. The present sensor utilises a section of fibre optic material which has been decladd or had the cladding removed. At this point, the exponentially decaying portion of the evanescent wave is harnessed to interact with the medium that surrounds it. In the sensor, the evanescent wave absorption phenomenon at the core cladding interface of an optical wave guide is used to determine different physical and chemical variables associated with microorganisms, and particularly bacterial, activity.

Typically an optical fibre is formed by making a preformed glass cylinder, drawing the fibres from the preform and testing the fibres. The glass for the preform is usually made by a process called modified chemical vapour deposition (MCBD). In MCBD, oxygen is bubbled through solutions of silicon chloride (SiCL₄), germanium chloride (GeCl₄) and/or other chemicals. The precise mixture governs the various physical and optical properties such as the index of refraction, coefficient of expansion and melting points. The gas vapours may then be conducted to the inside of a synthetic silica or quartz tube which forms the cladding. Under the effect of heat, silicon and germanium react to form silicon dioxide and germanium dioxide which deposit on the inside the tube and fuse together to form glass. A lathe is used to form an even coating and a consistent diameter. Purity of the glass may be maintained by using corrosion resistant plastic and in the gas delivery system, by precisely controlling the flow and composition of the mixture. The preformed blank is allowed to cool and is then loaded into a fibre drawing tower.

The blank may be lowered into a gravity furnace, which melts the tip until a pendulous blob falls down with gravity and forms a thread. This thread is then passed through a series of coating cups and ultra violet curing ovens to result in a product that has an inner core and outer cladding and often a buffer outer coating. The cladding is outer optical material surrounding the core and designed to reflect light back into the core. The buffer coating may be a plastic coating that protects the fibre from damage and moisture.

The fibres may be formed as single mode fibres which are used to transmit one signal per fibre or multi-mode fibres which may be used to transmit many signals per fibre. The single mode fibres have small cores of approximately 9 microns in diameter while multi-mode fibres have larger cores which may be up to 62.5 microns in diameter.

Cladding may be removed in a number of ways. One preferred procedure is a chemical etching process carried out by immersing the preferred region of the fibre in a 50% hydrofluoric acid solution for a period of 20 to 30 minutes.

In the biochemical recognition phase, a biochemical reaction which is unique to a microorganism or group of microorganisms is selected to identify the presence of the microorganisms. A precursor or transformable element of this reaction is selected for localisation in a coating placed over the denuded or declad optic fibre. Transformation of the precursor by microorganisms may result in a spectroscopically detectable indicator, the presence of which will be monitored by appropriate analysis means. Additionally, or alternatively, a specific indicator may also be selected wherein the transformed precursor will activate an adjunctive, co-operating indicator to provide a more easily detectable reaction.

In applying a coating to the optical fibre it is preferable to use a sol-gel technique. The sol-gel technique is utilised to form a porous, glass thin film coating around the cladding denuded optical fibre. Preparation of the sol-gel may be done at room temperature by the hydrolysis and condensation reaction of Tetra Ethyl Ortho Silica (TEOS) in an acidic environment, to form siloxane polymer, leading to gelation. The chemical reaction is as shown in the following equation:
Si(OC₂H₅)₄+₄H₂O→Si(OH)₄+4C₂H₅OH
Si(OH)₄→SiO2+2H₂O

The hydrolysis reaction of TEOS proceeds via the replacement of —OC₂H₅ groups by OH groups. Nominally, four H₂O molecules are required for the complete hydrolysis of a Si—(OC₂H₅)₄ molecules to form Si—(OH)₄ molecule.

The starting solution may be prepared by the partial hydrolysis of TEOS according to the above procedure. Denatured anhydrous ethanol, deionised water and hydrochloric acid may be used to perform the hydrolysis of TEOS. The entire mix may be maintained under constant stirring for 1 hour using a magnetic stirrer and then stored at room temperature. After 24 hours, the precursor solution and optical indicator may be mixed thoroughly into the material. This prepared sol may be used to coat the unclad portion of the optical fibre. Dip coating equipment may be used for this purpose.

The outer sol-gel coating displays three-dimensional porosities. The preferred pore size ranges from 10 nm to 100 nm. However, the preferred pore size may change with different applications. The porosity can be varied by: (1) altering the drying procedure of the sol-gel film, and (2) varying the molar ratios/chemical composition of the precursor chemicals.

In the spectroscopic analytical phase, absorption spectrometry may be used based on the fact that a given molecular species absorbs light in a specific region of the spectrum, and in varying degrees, that absorption pattern is characteristic of the particular species. In this system, the indicator or precursor is selected based on the products of metabolism or other chemical activity by microorganisms which display an absorption spectrum that may serve as a finger print for bacterial identification and monitoring purposes.

Spectroscopy is employed widely in laboratory diagnostics to study chemical processes such as metabolic reactions and enzyme kinetics amongst other things. Instruments reliant on spectroscopy make use of the absorption, emission or scattering of electromagnetic radiation in the examination of atoms or molecules of interest. In doing so, rapid qualitative and quantitative study of molecules has become possible hastening processes such as medical diagnostics and quality testing.

Briefly absorption spectroscopy utilises the transition between energy levels in molecules absorbing electromagnetic radiation. Ultraviolet and visible light incite electrons in atoms and molecules to higher energy levels and the amount of light energy absorbed is a function of the incident light wavelength. The unique absorption spectra displayed by different chemical species makes spectroscopy an indispensable tool in modern diagnostics.

Intensity of any given absorbent spectrum has a linear relationship with the concentration of the chemical species with that given absorption spectra. Hence, the levels of any given analyte can be rapidly determined using modern spectroscopic instruments thereby providing a quantitative indication of the amount of the material present.

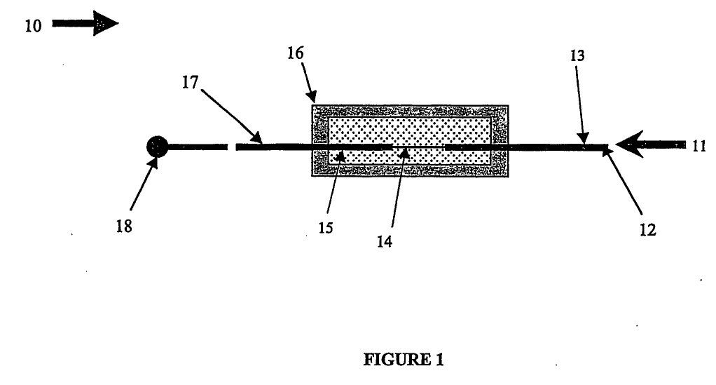

Referring to FIG. 1 there is seen a sensor system of the present invention generally designated as 10. The sensor system comprises a light source 11 which may conveniently provide light of a mixed and wide band length. The light is directed into an inlet end 12 of an optical fibre 13. A tungsten-halogen lamp may be used. Use of a photosensitive indicator in the present system, may result in output in the visible range. However, a xenon-arc lamp may also be used, especially when higher intensity and broader light spectrum is required. Other suitable light sources may be used.

A declad section 14 of the optical fibre 13 is provided with a coating. The coating immobilises a transformable precursor for a specific microorganism mediated reaction chosen for application. The coating is a bio-inert coating and may be preferably formed according to the above description as a thin film polymer layer. The coating may be permeable to microorganisms under review to facilitate interaction. The declad region 14 may be located in a specimen chamber 15 defined by an outer wall 16. The optical fibre 13 has a discharge end 17 for discharging light. The discharge end is adapted to cooperate with a spectrometer 18 which forms analysis means. In operation a sample such as saliva is located in the specimen chamber 15. Light is provided from the light source 11. At the declad region 14 active targeted cells, if present, metabolise these selected precursors to produce an indicator colour or other indicium such as an absorption pattern. The reaction may produce an indicator by activating a specific indicator which may be separately included in the outer coating as an adjunct to the transformable precursor.

The sensor system may produce an immediate response which may be monitored by a spectroscopic method in real time. The response, in some situations, will increase with time.

This method of using evanescent wave spectroscopy in conjunction with microorganisms and, in particular bacteria induced and mediated reaction to monitor microbial activity in, for example, body fluids provides a useful and quick indication of the presence of bacteria. The present sensor system process includes the sensor phase, the biochemical recognition phase and the signal transduction phase all in one optical fibre. It displays a very short response time and high sensitivity when used. This invention therefore introduces a combination of the benefits of, for example, established bacterial mediated chemical reaction identification and fibre optic spectroscopy. The invention produces a sensor with high sensitivity and specificity associated with an ability to rapidly identify bacteria and bacterial activity in real time.

A sensor of the present invention may be prepared as a probe for use in vivo or alternatively may be provided for ex Vivo application. While the below examples and discussion are directed to bacteria it should be understood that the sensor method may be turned to other types of microorganisms.

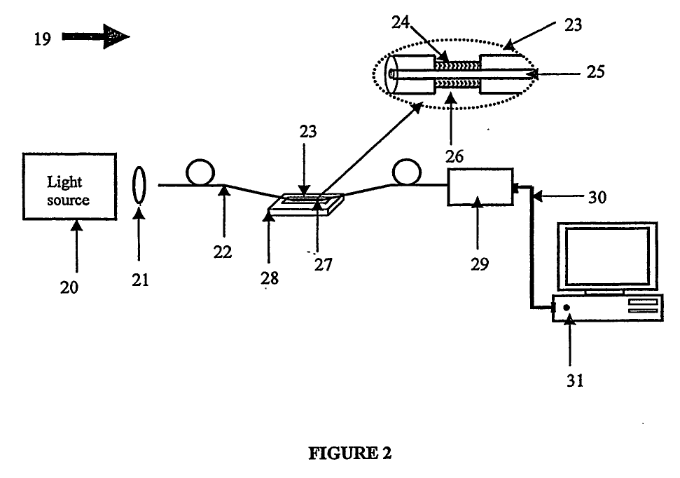

Referring to FIG. 2 there is seen a sensor system generally indicated as 19 incorporating a light source 20. A lens 21 may be effectively harnessed to regulate light output and the angle of incident rays. Light is delivered to a fibre optic member 22 which has a sensor element 23 which is also shown in close detail. The sensor element comprises a declad section 24 of a fibre optic core 25 and surface gel coating 26. The surface gel coating 26 is preferably formed as a bio-inert substance containing a selected biochemical precursor. The bacteria in a sample may interact with the precursor to produce optically detectable indicators. The sensor element 23 may be located in a channel 27 in a test pad 28. The sensor element 23 may be secured to the floor of the channel 27 using wedges so that a very small quantity of sample is required to adequately immerse the sensor portion. This embodiment of the device provides an easy accessible test region with a low requirement for quantity of specimen.

The detectable indicator may be optically detectable and produces light which continues along the fibre optic member 22 to a spectrometer 29. At this point, an inquiry is made of presented light and data is input through lead 30 to a processing means in the form of computer 31. Preferably the computer has a data store of information relating to characteristics of individual bacterial species or varieties. The computer may be programmed to compare incoming data against the data store to thereby provide an indication of the identity of bacteria present. Further the intensity and degree of the spectrometric results may result in an estimation of the quantitative concentration of bacteria in a sample.

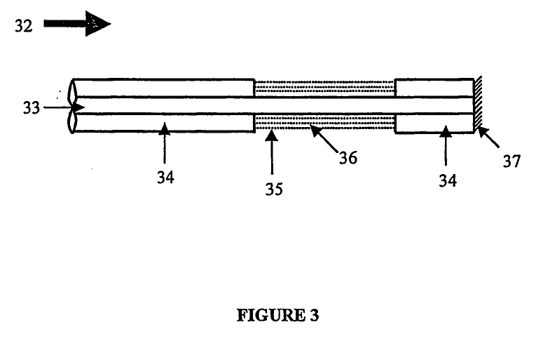

Referring to FIG. 3 there is seen a probe 32 for a sensor system of the present invention comprising an inner fibre optic core 33 having an external cladding 34. The cladding is removed in a sensor zone 35 and replaced by an external coating 36 of a bio-inert material which includes a preselected precursor for metabolism by bacteria of interest. The interrupted cladding continues 34 and the cladding 34 and inner core 33 are terminated by a reflecting surface 37 which reflects incident light back up the fibre optic core for analysis. The probe is particularly useful as it may be inserted into cavities of a patient or into other samples which may be difficult to access or which may be toxic. The reflected light may be subsequently analysed according to the above description.

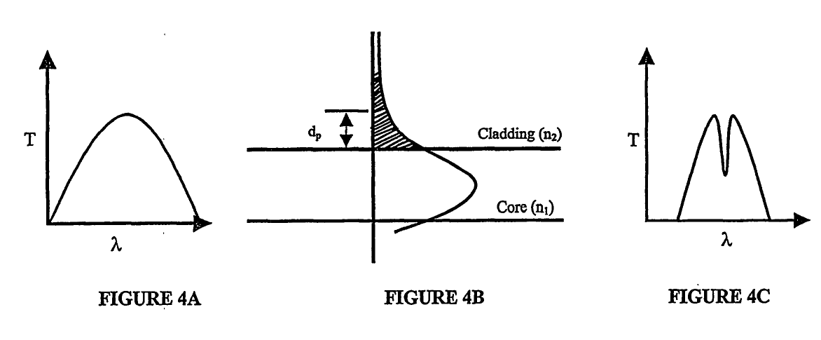

The light source produces a transmission spectrum as shown in FIG. 4A (depending on the nature of light) wherein wavelength (λ) is plotted against intensity (T). The evanescent wave distribution at the core-cladding interface can be represented as in FIG. 4B. It has a maximum intensity in proximity to the core and a taper in intensity away from the core. When a photosensitive indicator is immobilized within a porous class coating at the cladding denuded optical fiber, subsequently the transmission spectrum obtained is as shown in FIG. 4C. The valley in the transmission spectrum is due to the absorbance of the photosensitive indicator at a specific wavelength.

The power transmission in an optical fiber, having an absorbing cladding, is given by the modified Beer-Lambert's law:
P(I)=P₀exp(λI) (1)
where I is the distance along the unclad portion of the fiber, P0 is the power transmitted in the absence of an absorbing species and (is the evanescent wave absorption coefficient.
The above equation can be rewritten as,
P(I)=P₀exp(rαI) (2)
r is the fraction of the power transmitted through the cladding and (is the bulk absorption coefficient of the cladding. The evanescent wave absorbance ‘A’ from the previous equations as log P0/P(I).

$$A = \frac{γ l}{2.303} = \frac{r α l}{2.303} \qquad ( 3 )$$

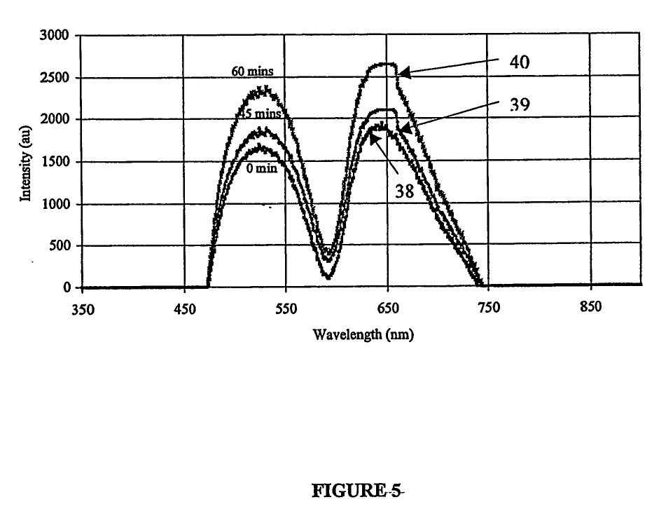

FIG. 5 shows a graph demonstrating another example of another example of results from the use of the biosensor. Two peaks are provided at proximity 480 and 640 nanometer wavelengths. Increasing results as shown between 0 minutes, 45 minutes and 60 minutes as plots 38, 39 and 40, respectively. The pattern may be specific for a transformed precursor or associated indicator. Presence of this indicator may be conclusive evidence of the present of live and active bacteria of a type under investigation.

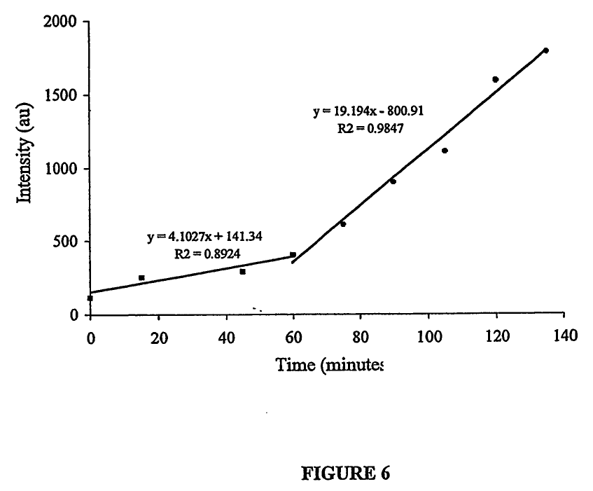

FIG. 6 is a typical graph representing the activity profile of streptococcus mutans with sucrose in human saliva monitored by the present fibre optic sensor. This graph represents the increase in the formation of extra cellular polysacchride and lactic acid adjacent or on the sensor from 5 minutes to 120 minutes time duration. The increase in by-product formation with time show two slopes. In the first phase, the slope is significantly smaller than the second phase. This denotes increasing activity (greater concentration) of the by-product formation in the second phase compared to that of the first. The change was detected by the fibre optic sensor system of the present invention.

It is clear to the skilled addressee that multiple sensor sections may be used on one single fibre optic member. Such sensor sections may be contiguous or alternatively may be separated by areas of clad central core. In a further alternative, multiple fibre optic members may be used. These multiple members may be arranged substantially parallel and may produce a bank or array of sensors to provide multiple results.

### EXAMPLE 1

### Staphylococcus aureus

Staphylococcus Aureus is a pathogenic bacterium that causes significant morbidity particularly to immune compromised individuals. It is also a common food borne bacterium. Nosocomial infections due to this bacterium create problems that are increasing in severity and are a financial and health liability in the clinical environment. It has become important to develop a rapid detection system for Staphylococcus aureus and in particular methicillin resistant Staphylococcus aureus.

A conventional growth and indication medium for the detection of Staphylococcus aureus is mannitol salt agar, which is both a selective and differential growth medium. It is used to differentiate pathogenic Staphylococcus species from non-pathogenic members of the genus Micrococcus. The medium typically contains about 7.5% salt thus selecting for organisms that are able to tolerate the presence of high levels of salt. This medium also contains an indicator, phenol red, which is a pinkish red at neutral pH, red at pH at 7.4 and above and is yellow below the pH 6.8. Organisms that ferment mannitol produce acid as a reaction product hence causing colour change to the indicator.

A sample containing Staphylococcus aureus was placed in contact with the sensor portion of the present invention. Production of acid from D-mannitol in the presence of methicillin resulted in an optical indicator, which was detected by spectroscopic means, providing a distinctive absorption spectrum.

### EXAMPLE 2

### Mycobacterium

The infectious agents of tuberculosis and leprosy belong to the same bacterial genus, Mycobacterium. Infection by Mycobacterium tuberculosis causes fever, cough, loss of energy and weight loss and serious lung damage. Leprosy, an infection of the skin, peripheral nerves and mucous membranes is caused by Mycobacterium leprae. The serious nature of these mycobacteria makes the need for a rapid diagnosis necessary to ensure appropriate therapeutic treatment can be initiated as early as possible.

A sample containing Mycobacterium leprae and Mycobacterium tuberculosis was placed in contact with the sensor element of the present system. Carbol fuchsine was dispersed in the outer coating of the sensor element. Reaction of carbol fuchsine with lipids of the mycobacterium cell wall produced an absorption pattern which was distinctive and indicated the presence of the mycobacteria.

### EXAMPLE 3

### Environmental Sampling

General bacterial contamination in the environment may be identified using the system of the present invention. Reaction with general dyes such as methylene blue and features of bacteria such as the cell wall may produce an optically or spectroscopically detectable indicator.

A contaminated sample with mixed bacterial population was placed in contact with the sensor element of the present invention. A general absorption pattern was detected by spectroscopic methods to indicate the presence of multiple bacterial organisms.

### EXAMPLE 4

### Staphylococcus mutans

Staphylococcus mutans is a reliable indicator of a predisposition to dental caries. The outer coating of the sensor element of the present system was impregnated with sucrose. The sensor element was brought into contact with a sample containing Staphylococcus mutans which resulted in metabolism of sucrose to form latic acid and polysaccharides, thereby resulting in an optically detectable indicator.

In a further version of this example, bacitracin was also mixed with the sample to render the test more specific for Staphylococcus mutans.

In one embodiment the system may include data processing means. The data processing means may assign numerical values to certain characteristics of identified microorganisms and in particular bacteria. The assigning of numerical values enables data processing means to assess the status of a sample such as a biological or environmental sample. Data processing may result in a quantitative indicator of the status of contamination of a sample or alternatively may provide a generic result, such as “low” or “moderate” and “high” contamination levels.

The types of attributes which may be ascribed a numerical value include bacterial genus, bacterial species, bacterial variety, bacterial concentration and rate of development of change of indicator.

The value ascribed to each feature may be referred to as an index value (Iᵥ).

The sum of Iᵥ, ie. ΣIᵥ, provides a contamination index of a sample (C_{I}) value and this enables an analytical approach to screening and identifying contamination of samples. Clearly the process may be equally directed to identifying the health or presence of commensal organisms and normal healthy flora in certain samples. The (Iᵥ) index value for each feature may be stored in a machine readable storage program, which may be capable of processing the data to provide a contamination value for a sample or group of samples.

Thus, in another aspect, the invention contemplates a computer program product for assessing the status of presence of microorganisms of a sample or group of samples, said product comprising:

code that receives as input index values for at least two features associated with microorganisms where in the features are selected from a group including:

a) genus of microorganism;

b) species of microorganism;

c) variety of microorganisms;

d) concentration of microorganisms; and

e) speed of development of indicators.

a code that adds the index values to provide a sum corresponding to a contamination index for the sample; and

a computer readable medium that stores the code.

In a preferred embodiment, the computer program product comprises code that assigns an index value for each feature of the microorganism or group of microorganisms.

In a related aspect, the invention extends to a computer for assessing the likelihood of contamination of a sample compound or group of samples wherein the computer comprises:

a machine readable data storage medium comprising a data storage material encoded with machine readable data, wherein said machine readable data comprise index values for at least two features associated with microorganisms wherein the features are selected from:

a) genus of microorganism;

b) species of microorganism;

c) variety of microorganisms;

d) concentration of microorganisms; and

e) speed of development of indicators;

a working memory for storing instructions for processing the machine readable data;

a central processing unit coupled to the working memory and to the machine readable data storage medium, for processing the machine readable data to provide a sum of the index values corresponding to a potency value for the compounds; and

an output hardware coupled to the central processing unit for receiving the contamination values.

A version of these embodiments may be represented in a figure which shows a system including a computer (31, FIG. 2) comprising a central processing unit (“CPU”), a working memory which may be, for example, RAM (Random Access Memory) or “Core” memory, mass storage memory such as one or more disk drives or CD ROM drives, one or more cathode ray tube display terminals, one or more keyboards, one or more input lines and one or more output lines all of which are interconnected by a conventional bi-directional system bus.

Input hardware may be coupled to the computer by input lines which may be implemented in a variety of ways. For example, machine readable data of this invention may be inputted by the use of a modem or modems connected by a telephone line or dedicated data line. Alternatively or additionally, the input hardware may comprise a CD. Alternatively, ROM drives or disk drives in conjunction with display terminals, keyboards and keyboard may also be used as a input device. The output device, coupled to a computer by output lines, may similarly be implemented by conventional devices. Output hardware might also include a printer so that hard copy output may be produced, or a disk drive to store system output for later use.

In operation the CPU coordinates the use of the various input and output devices coordinates data accesses from our storage and accesses to and from working memory and determines the sequence of data processing. A number of programs may be used to process the machine readable data of this invention.

The computer is located in signal connection with a spectroscope for receiving and analysing input to produce at least one indicator of the microorganism status of a sample.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the disclosure.

The claims defining the invention are as follows:

1. A sensor for sensing and/or monitoring at least one property associated with transformation of a biochemical analyte by at least one microorganism, said sensor comprising:
at least one fibre optic member having at least one unclad portion;
a coating applied to the at least one unclad portion;
a precursor associated with the coating, said precursor transformable by the at least one microorganism;
wherein
transformation of the precursor produces a spectroscopically detectable indicator of the at least one property.

2. The sensor of claim 1 wherein the unclad portion of the fibre optic member is a declad portion.

3. The sensor of claim 1 comprising a plurality of unclad portions.

4. The sensor of claim 3 further comprising two or more separate fibre optic members.

5. The sensor of claim 1 further adapted to cooperate with analysis means for determining the presence of the spectroscopically detectable indicator.

6. The sensor of any one of the proceeding claims wherein the coating is a glass film.

7. The sensor of the proceeding claim wherein the glass film is both porous and thin.

8. The sensor of any one of the proceeding claims wherein the precursor is immobilised within the coating.

9. The sensor of the proceeding claim wherein the precursor comprises one or more of D-mannitol, carbol fuchsine, methylene blue, sucrose or other suitable compound.

10. The sensor of claim 1 wherein transformation of the precursor results in a product which cooperates with an adjunctive compound to produce the spectroscopically detectable indicator.

11. A sensor system for sensing at least one property associated with transformation of a precursor by one or more microorganisms, said sensor system comprising:
a fibre optic member having at least one unclad portion of optic fibre;
a coating applied to the at least one unclad portion;
a precursor associated with the coating, said precursor transformable by at the one or more microorganisms;
a light source adapted to cooperate with a first end of the fibre optic member to provide input light to the fibre optic member; and
monitoring means adapted to cooperate with the unclad portion to detect an indicator signal in received light from the fibre optic member, said indicator signal indicative of the at least one property;
wherein
transformation of the precursor by the one or more microorganisms produces the indicator signal by interaction with the input light to produce the received light.

12. The sensor system of claim 12 wherein interaction with the light is interactive with an evanescent wave form of the input light.

13. A method of producing a sensor, said method comprising the steps of:
decladding one or more sections of a core of a fibre optic member;
applying a coating to the one or more sections, said coating immobilising a precursor to a spectroscopically detectable indicator, the precursor transformable to the detectable indicator by the activity of one or more microorganisms.

14. A method of identifying the presence of at least one type of microorganism comprising the steps of:
activating a light source in cooperating relationship to a first end of a sensor according to any one of claims 1 to 11;
monitoring the electromagnetic output from a coated unclad section;
locating the sensor with its coated unclad section in contact with the sample; and
analysing the electromagnetic output to determine the presence of the at least one type of microorganism.

15. The method of claim 14 wherein monitoring the electromagnetic output comprises spectroscopically monitoring the electromagnetic output.

16. The method of either one of claim 14 or claim 15 wherein analysing the electromagnetic output comprises conducting absorption analysis to identify wave lengths of peak absorption of electromagnetic output.

17. The method of claim 16 wherein analysis of the electromagnetic output includes operating a programmable device programmed to receive digital information from a spectroscope and provide an analysis of results.

18. The method of claim 17 wherein the programmable device is programmed to identify one or more features of the at least one microorganism, the one or more features being selected from a group including genus of microorganism, species of microorganism, variety of microorganism, concentration of microorganism and speed of development of indicator.

19. The method of claim 18 wherein the programmable device is further programmed to ascribe an index value to each identified feature and provide an overall index for a sample according to the algorithm
Cₛ=ΣIv
where:
C=and overall index
Iv=individual indices.

20. A method of coating a sensor for sensing and/or monitor at least one property associated with transformation of a biochemical analyte by at least one microorganism, comprising steps of:
making a coating mixture by dispersing a precursor in a sol-gel solution; wherein the precursor is transformable by the at least one microorganism; and
coating the sensor with the coating mixture; wherein the sensor comprises at least one fiber optic member having at least one unclad portion, and the coating is preferably applied to the unclad portion.

21. The method of coating a sensor of claim 20, wherein the sol-gel solution is made by hydrolysis of Tetra Ethyl Ortho Silica; wherein the molecule used for hydrolysis is selected from the group consisting of H₂O, anhydrous ethanol, and hydrochloric acid.

22. The method of coating a sensor of any of claims 20-21, wherein the sol-gel coating is done by dip coating.

23. The method of coating a sensor of any of claims 20-22, wherein the precursor is selected from the group consisting of D-mannitol, carbol fuchsine, methylene blue, and sucrose.

24. The method of costing a sensor of any claims 20-23, wherein the resultant product from the transformable precursor cooperates with an adjunctive compound to produce the spectroscopically detectable indicator.
